(19) United States
(12) Patent Application Publication
(10) Pub. No.: US 2017/0312264 A1
(43) Pub. Date: Nov. 2, 2017

(54) USE OF LAQUINIMOD TO DELAY HUNTINGTON'S DISEASE PROGRESSION

(71) Applicants: Anna Kristina Sveinsdotter Teige Wickenberg, Lund (SE); Esther Lukasiewicz Hagai, Tel Aviv (IL); Eli Eyal, Petach-Tikva (IL); Sigal Melamed-Gal, Chalfont, PA (US)

(72) Inventors: Anna Kristina Sveinsdotter Teige Wickenberg, Lund (SE); Esther Lukasiewicz Hagai, Tel Aviv (IL); Eli Eyal, Petach-Tikva (IL); Sigal Melamed-Gal, Chalfont, PA (US)

(73) Assignee: Teva Pharmaceutical Industries, Ltd., Petach-Tikva (IL)

(21) Appl. No.: 15/653,132

(22) Filed: Jul. 18, 2017

Related U.S. Application Data
(63) Continuation of application No. 15/479,435, filed on Apr. 5, 2017.
Application No. 15/479,435 is a continuation of application No. 15/388,947, filed on Dec. 22, 2016.
Application No. 15/388,947 is a continuation of application No. 14/575,357, filed on Dec. 18, 2014, now abandoned.
(60) Provisional application No. 61/919,604, filed on Dec. 20, 2013.

(51) Int. Cl. A61K 31/47; A61K 9/20

(52) U.S. Cl. CPC A61K 31/47; A61K 9/2054; A61K 9/20; A61K 9/2004; A61K 9/2013

(57) ABSTRACT

The subject invention provides methods of treating or delaying disease progression in a subject afflicted with Huntington's disease (HD) comprising administering to the subject 0.5-1.5 mg/day laquinimod. The subject invention also provides packages, therapeutic packages and pharmaceutical compositions, comprising one or more unit doses of 0.5-1.5 mg laquinimod for treating or delaying disease progression in a subject afflicted with HD. Also disclosed is use of laquinimod in the manufacture of a medicament comprising one or more unit doses of 0.5-1.5 mg laquinimod for use in treating or delaying disease progression in a subject afflicted HD.

This application claims benefit of U.S. Provisional Application No. 61/919,604, filed Dec. 20, 2013, the entire content of which is hereby incorporated by reference herein.

Throughout this application, various publications are referred to by first author and year of publication. Full citations for these publications are presented in a References section immediately before the claims. Disclosures of the documents and publications referred to herein are hereby incorporated in their entireties by reference into this application.

### BACKGROUND

### Huntington's Disease (HD)

HD is an autosomal dominant neurodegenerative disorder characterized by motor, cognitive, behavioral, functional and psychiatric symptoms and by a progressive degeneration of neurons in basal ganglia in brain cortex. (Huntington Study Group, 1996; Ciammola, 2007).

### Laquinimod

Laquinimod (LAQ) is a novel synthetic compound with high oral bioavailability which has been suggested as an oral formulation for the treatment of Multiple Sclerosis (MS) (Polman, 2005; Sandberg-Wollheim, 2005). Laquinimod and its sodium salt form are described, for example, in U.S. Pat. No. 6,077,851. The mechanism of action of laquinimod is not fully understood. Animal studies show it causes a Th1 (T helper 1 cell, which produces pro-inflammatory cytokines) to Th2 (T helper 2 cell, which produces anti-inflammatory cytokines) shift with an anti-inflammatory profile (Yang, 2004; Brück, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the Central Nervous System (CNS), increase of axonal integrity, modulation of cytokine production, and increase in levels of brain-derived neurotrophic factor (BDNF) (Runström, 2006; Brück, 2011).

The effect of laquinimod in delaying disease progression in Huntington's disease patients was not previously reported.

### SUMMARY OF THE INVENTION

The subject invention provides a method of delaying disease progression in a subject afflicted with Huntington's disease comprising administering to the subject 0.5-1.5 mg/day of laquinimod thereby delaying disease progression in the subject.

The subject invention also provides a method of treating a subject afflicted with Huntington's disease comprising administering to the subject an amount of laquinimod so as to thereby treat the subject, wherein the amount laquinimod administered is selected from the group consisting of 0.5 mg/day, 1.0 mg/day and 1.5 mg/day.

The subject invention also provides a package comprising: a) a pharmaceutical composition comprising one or more unit doses, each such unit dose comprising 0.5-1.5 mg of laquinimod; and b) instruction for use of the pharmaceutical composition to delay disease progression in a subject afflicted with Huntington's disease.

The subject invention also provides a package comprising: a) a pharmaceutical composition comprising one or more unit doses, each such unit dose comprising 0.5, 1.0 or 1.5 mg of laquinimod; and b) instruction, for use of the pharmaceutical composition to treat a subject afflicted with Huntington's disease.

The subject invention also provides a therapeutic package for dispensing to, or for use in dispensing to, a subject afflicted with Huntington's disease, which comprises: a) one or more unit doses, each such unit dose comprising 0.5-1.5 mg of laquinimod, and b) a finished pharmaceutical container therefor, said container containing said unit dose or unit doses, said container further containing or comprising labeling directing the use of said package in delaying disease progression in said subject.

The subject invention also provides a therapeutic package for dispensing to, or for use in dispensing to, a subject afflicted with Huntington's disease, which comprises: a) one or more unit doses, each such unit dose comprising 0.5 mg, 1.0 mg or 1.5 mg of laquinimod, and b) a finished pharmaceutical container therefor, said container containing said unit dose or unit doses, said container further containing or comprising labeling directing the use of said package in treating said subject.

The subject invention also provides a pharmaceutical composition comprising one or more unit doses, each such unit dose comprising 0.5-1.5 mg of laquinimod, for use in delaying disease progression in a subject afflicted with Huntington's disease.

The subject invention also provides a pharmaceutical composition comprising one or more unit doses, each such unit dose comprising 0.5 mg, 1.0 mg and 1.5 mg of laquinimod, for use in treating a subject afflicted with Huntington's disease.

The subject invention also provides a package comprising any of the pharmaceutical compositions described herein and instruction for use of the pharmaceutical composition to treat or delay disease progression in a subject afflicted with Huntington's disease.

The subject invention also provides laquinimod for the manufacture of a medicament for use in delaying disease progression in a subject afflicted Huntington's disease, wherein the medicament comprises one or more unit doses, each such unit dose comprising 0.5-1.5 mg of laquinimod.

The subject invention also provides laquinimod for the manufacture of a medicament for use in treating a subject afflicted Huntington's disease, wherein the medicament comprises one or more unit doses, each such unit dose comprising 0.5, 1.0 or 1.5 mg of laquinimod.

### DETAILED DESCRIPTION OF THE INVENTION

Treatment of a human patient suffering from a brain-derived neurotrophic factor (BDNF)-related disease by periodic administration of laquinimod is disclosed in U.S. Application Publication No. US 2011/0034508. US 2011/0034508 further teaches that HD is “a BDNF-related disease”.

While one having ordinary skill in the art may expect laquinimod to exhibit some therapeutic activity in HD based on the teaching of US 2011/0034508, the instant invention is directed to an improved treatment. Specifically, the inventors have surprisingly found that 0.5-1.5 mg/day laquinimod is especially effective in delaying progression of disease progression, particularly in symptomatic early HD patients.

The subject invention provides a method of delaying disease progression in a subject afflicted with Huntington's disease comprising administering to the subject 0.5-1.5 mg/day of laquinimod thereby delaying disease progression in the subject. In an embodiment, the amount laquinimod administered is selected from the group consisting of 0.5 mg/day, 1.0 mg/day and 1.5 mg/day.

The subject invention also provides a method of treating a subject afflicted with Huntington's disease comprising administering to the subject an amount of laquinimod so as to thereby treat the subject, wherein the amount laquinimod administered is selected from the group consisting of 0.5 mg/day, 1.0 mg/day and 1.5 mg/day.

In an embodiment, the amount laquinimod administered is 0.5 mg/day. In another embodiment, the amount laquinimod administered is 1.0 mg/day. In another embodiment, the amount laquinimod administered is 1.5 mg/day.

In one embodiment, the subject is afflicted with adult onset Huntington's disease. In another embodiment, the subject has a Unified Huntington's Disease Rating Scale (UHDRS)−Total Motor Score (TMS) of greater than 5 at baseline. In another embodiment, the subject has Unified Huntington's Disease Rating Scale (UHDRS)−Total Functional Capacity (TFC) score of at least 8 at baseline. In another embodiment, the subject is ambulatory at baseline. In another embodiment, the subject is naïve to a Huntington's disease therapy at baseline. In another embodiment, the subject is naïve to any Huntington's disease therapy at baseline. In yet another embodiment, the subject is naïve to laquinimod at baseline.

In an embodiment, the subject is determined to have ≧36 cytosine-adenosine-guanine (CAG) repeats in the huntingtin gene. In another embodiment, the subject is determined to have 40-49 cytosine-adenosine-guanine (CAG) repeats in the huntingtin gene.

In an embodiment, laquinimod is laquinimod sodium. In another embodiment, laquinimod is administered via oral administration. In another embodiment, laquinimod is administered periodically or daily. In another embodiment, laquinimod is administered daily at the same time of the day. In another embodiment, laquinimod is administered periodically for 12 months or more.

In one embodiment, the method as described herein further comprises administration of a second agent for the treatment of Huntington's disease.

The subject invention also provides a package comprising: a) a pharmaceutical composition comprising one or more unit doses, each such unit dose comprising 0.5-1.5 mg of laquinimod; and b) instruction for use of the pharmaceutical composition to delay disease progression in a subject afflicted with Huntington's disease. In an embodiment, the amount of laquinimod in the pharmaceutical composition is selected from the group consisting of 0.5 mg, 1.0 mg and 1.5 mg.

The subject invention also provides a package comprising: a) a pharmaceutical composition comprising one or more unit doses, each such unit dose comprising 0.5, 1.0 or 1.5 mg of laquinimod; and b) instruction for use of the pharmaceutical composition to treat a subject afflicted with Huntington's disease.

In an embodiment, the package comprises a second pharmaceutical composition comprising an amount of a second agent for the treatment of Huntington's disease. In another embodiment, the pharmaceutical composition is in a solid or liquid form. In another embodiment, the pharmaceutical composition is in capsule form or in tablet form.

In an embodiment, the tablet is coated with a coating which inhibits oxygen from contacting the core. In another embodiment, the coating comprises a cellulosic polymer, a detackifier, a gloss enhancer, or pigment.

In an embodiment, the pharmaceutical composition further comprises mannitol. In another embodiment, the pharmaceutical composition further comprises an alkalinizing agent. In another embodiment, the alkalinizing agent is meglumine. In another embodiment, the pharmaceutical composition further comprises an oxidation reducing agent.

In one embodiment, the pharmaceutical composition is stable and free of an alkalinizing agent or an oxidation reducing agent. In another embodiment, the pharmaceutical composition is free of an alkalinizing agent and free of an oxidation reducing agent. In another embodiment, the pharmaceutical composition is stable and free of disintegrant.

In one embodiment, the pharmaceutical composition further comprises a lubricant. In another embodiment, the lubricant is present in the pharmaceutical composition as solid particles. In another embodiment, the lubricant is sodium stearyl fumarate or magnesium stearate. In another embodiment, the pharmaceutical composition further comprises a filler. In another embodiment, the filler is present in the pharmaceutical composition as solid particles. In another embodiment, the filler is lactose, lactose monohydrate, starch, isomalt, mannitol, sodium starch glycolate, sorbitol, lactose spray dried, lactose anhydrouse, or a combination thereof. In another embodiment, the filler is mannitol or lactose monohydrate.

In one embodiment, the package further comprises a desiccant. In another embodiment, the desiccant is silica gel.

In one embodiment, the pharmaceutical composition is stable and has a moisture content of no more than 4%. In another embodiment, laquinimod is present in the pharmaceutical composition as solid particles.

In one embodiment, the package is a sealed packaging having a moisture permeability of not more than 15 mg/day per liter. In another embodiment, the sealed package is a blister pack in which the maximum moisture permeability is no more than 0.005 mg/day. In another embodiment, the sealed package is a bottle. In another embodiment, the bottle is closed with a heat induction liner. In another embodiment, the sealed package comprises an HDPE bottle. In another embodiment, the sealed package comprises an oxygen absorbing agent. In another embodiment, the oxygen absorbing agent is iron.

In one embodiment, the pharmaceutical composition is formulated for oral administration. In another embodiment, the pharmaceutical composition is formulated for daily administration. In another embodiment, the package is prepared for use in treating or delaying disease progression in a subject afflicted with Huntington's disease.

The subject invention also provides a therapeutic package for dispensing to, or for use in dispensing to, a subject afflicted with Huntington's disease, which comprises: a) one or more unit doses, each such unit dose comprising 0.5-1.5 mg of laquinimod, and b) a finished pharmaceutical container therefor, said container containing said unit dose or unit doses, said container further containing or comprising labeling directing the use of said package in delaying disease progression in said subject. In one embodiment, each unit dose comprises an amount of laquinimod selected from the group consisting of 0.5 mg, 1.0 mg and 1.5 mg.

The subject invention also provides a therapeutic package for dispensing to, or for use in dispensing to, a subject afflicted with Huntington's disease, which comprises: a) one or more unit doses, each such unit dose comprising 0.5 mg, 1.0 mg or 1.5 mg of laquinimod, and b) a finished pharmaceutical container therefor, said container containing said unit dose or unit doses, said container further containing or comprising labeling directing the use of said package in treating said subject.

In an embodiment of any of the packages disclosed herein, the package comprises an amount of a second agent for the treatment of Huntington's disease.

The subject invention also provides a pharmaceutical composition comprising one or more unit doses, each such unit dose comprising 0.5-1.5 mg of laquinimod, for use in delaying disease progression in a subject afflicted with Huntington's disease. In one embodiment, the pharmaceutical composition comprises an amount of laquinimod selected from the group consisting of 0.5 mg, 1.0 mg and 1.5 mg.

The subject invention also provides a pharmaceutical composition comprising one or more unit doses, each such unit dose comprising 0.5 mg, 1.0 mg and 1.5 mg of laquinimod, for use in treating a subject afflicted with Huntington's disease.

In one embodiment, the pharmaceutical composition further comprises an amount of a second agent for the treatment of Huntington's disease.

In one embodiment, laquinimod is laquinimod sodium. In another embodiment, the pharmaceutical composition is in a solid or liquid form. In another embodiment, the pharmaceutical composition is in capsule form or in tablet form. In another embodiment, the tablet is coated with a coating which inhibits oxygen from contacting the core. In another embodiment, the coating comprises a cellulosic polymer, a detackifier, a gloss enhancer, or pigment.

In one embodiment, the pharmaceutical composition further comprises mannitol. In another embodiment, the pharmaceutical composition further comprises an alkalinizing agent. In an embodiment, the alkalinizing agent is meglumine.

In one embodiment, the pharmaceutical composition further comprises an oxidation reducing agent. In another embodiment, the pharmaceutical composition is free of an alkalinizing agent or an oxidation reducing agent. In another embodiment, the pharmaceutical composition is free of an alkalinizing agent and free of an oxidation reducing agent.

In one embodiment, the pharmaceutical composition is stable and free of disintegrant. In another embodiment, the pharmaceutical composition further comprises a lubricant. In another embodiment, the lubricant is present in the pharmaceutical composition as solid particles. In another embodiment, the lubricant is sodium stearyl fumarate or magnesium stearate.

In one embodiment, the pharmaceutical composition further comprises a filler. In another embodiment, the filler is present in the pharmaceutical composition as solid particles. In another embodiment, the filler is lactose, lactose monohydrate, starch, isomalt, mannitol, sodium starch glycolate, sorbitol, lactose spray dried, lactose anhydrouse, or a combination thereof. In another embodiment, the filler is mannitol or lactose monohydrate.

In one embodiment, the pharmaceutical composition is formulated for oral administration. In another embodiment, the pharmaceutical composition is formulated for daily administration.

The subject invention also provides a package comprising any of the pharmaceutical compositions described herein and instruction for use of the pharmaceutical composition to treat or delay disease progression in a subject afflicted with Huntington's disease.

The subject invention also provides laquinimod for the manufacture of a medicament for use in delaying disease progression in a subject afflicted Huntington's disease, wherein the medicament comprises one or more unit doses, each such unit dose comprising 0.5-1.5 mg of laquinimod. In one embodiment, each such unit dose comprises 0.5 mg, 1.0 mg or 1.5 mg laquinimod.

The subject invention also provides laquinimod for the manufacture of a medicament for use in treating a subject afflicted Huntington's disease, wherein the medicament comprises one or more unit doses, each such unit dose comprising 0.5, 1.0 or 1.5 mg of laquinimod.

For the foregoing embodiments, each embodiment disclosed herein is contemplated as being applicable to each of the other disclosed embodiments. For instance, the elements recited in the method embodiments can be used in the pharmaceutical composition, package, and use embodiments described herein and vice versa.

### Terms

As used herein, and unless stated otherwise, each of the following terms shall have the definition set forth below.

As used herein, “laquinimod” means laquinimod acid or a pharmaceutically acceptable salt thereof.

A “salt thereof” is a salt of the instant compounds which have been modified by making acid or base salts of the compounds. The term “pharmaceutically acceptable salt” in this respect, refers to the relatively non-toxic, inorganic and organic acid or base addition salts of compounds of the present invention.

For example, one means of preparing such a salt is by treating a compound of the present invention with an inorganic base.

As used herein, an “amount” or “dose” of laquinimod as measured in milligrams refers to the milligrams of laquinimod acid, present in a preparation, regardless of the form of the preparation. A “dose of 0.5 mg laquinimod” means the amount of laquinimod acid in a preparation is 0.5 mg, regardless of the form of the preparation. Thus, when in the form of a salt, e.g. a laquinimod sodium salt, the weight of the salt form necessary to provide a dose of 0.5 mg laquinimod would be greater than 0.5 mg (e.g., 0.534 mg) due to the presence of the additional salt ion.

As used herein, a “unit dose”, “unit doses” and “unit dosage forms(s)” mean a single drug administration entity/entities.

As used herein, “about” in the context of a numerical, value or range means ±10% of the numerical value or range recited or claimed.

As used herein, a composition that is “free” of a chemical entity means that the composition contains, if at all, an amount of the chemical entity which cannot be avoided although the chemical entity is not part of the formulation and was not affirmatively added during any part of the manufacturing process. For example, a composition which is “free” of an alkalizing agent means that the alkalizing agent, if present at all, is a minority component of the composition by weight. Preferably, when a composition is “free” of a component, the composition comprises less than 0.1 wt %, 0.05 wt %, 0.02 wt %, or 0.01 wt % of the component.

As used herein, “alkalizing agent” is used interchangeably with the term “alkaline-reacting component” or “alkaline agent” and refers to any pharmaceutically acceptable excipient which neutralizes protons in, and raises the pH of, the pharmaceutical composition in which it is used.

As used herein, “oxidation reducing agent” refers to a group of chemicals which includes an “antioxidant”, a “reduction agent” and a “chelating agent”.

As used herein, “antioxidant” refers to a compound selected from the group consisting of tocopherol, methionine, glutathione, tocotrienol, dimethyl glycine, betaine, butylated hydroxyanisole, butylated hydroxytoluene, turmerin, vitamin E, ascorbyl palmitate, tocopherol, deteroxime mesylate, methyl paraben, ethyl paraben, butylated hydroxyanisole, butylated hydroxytoluene, propyl gallate, sodium or potassium metabisulfite, sodium or potassium sulfite, alpha tocopherol or derivatives thereof, sodium ascorbate, disodium edentate, BHA (butylated hydroxyanisole), a pharmaceutically acceptable salt or ester of the mentioned compounds, and mixtures thereof.

The term “antioxidant” as used herein also refers to Flavonoids such, as those, selected from the group of quercetin, morin, naringenin and hesperetin, taxifolin, afzelin, quercitrin, myricitrin, genistein, apigenin and biochanin A, flavone, flavopiridol, isoflavonoids such as the soy isoflavonoid, genistein, catechins such as the tea catechin epigallocatechin gallate, flavonol, epicatechin, hesperetin, chrysin, diosmin, hesperidin, luteolin, and rutin.

As used herein, “reduction agent” refers to a compound selected from the group consisting of thiol-containing compound, thioglycerol, mercaptoethanol, thioglycol, thiodiglycol, cysteine, thioglucose, dithiothreitol (DTT), dithio-bis-maleimidoethane (DTME), 2,6-di-tert-butyl-4-methylphenol (BHT), sodium dithionite, sodium bisulphite, formamidine sodium metabisulphite, and ammonium bisulphite.”

As used herein, “chelating agent” refers to a compound selected from the group consisting of penicillamine, trientine, N,N′-diethyldithiocarbamate (DDC), 2,3,2′-tetraamine (2,3,2′-tet), neocuproine, N,N,N′,N′-tetrakis (2-pyridylmethyl) ethylenediamine (TPEN), 1,10-phenanthroline (PHE), tetraethylenepentamine, triethylenetetraamine and tris(2-carboxyethyl) phosphine (TCEP), ferrioxamine, CP94, EDTA, deferoxainine B (DFO) as the methanesulfonate salt (also known as desferrioxanilne B mesylate (DFOM)), desferal from Novartis (previously Ciba-Giegy), and apoferritin.

As used herein, a pharmaceutical composition is “stable” when the composition preserves the physical stability/integrity and/or chemical stability/integrity of the active pharmaceutical ingredient during storage. Furthermore, “stable pharmaceutical composition” is characterized by its level of degradation products not exceeding 5% at 40° C./75% RH after 6 months or 3% at 55° C./75% RH after two weeks, compared to their level in time zero.

“Treating” as used herein encompasses, e.g., inducing inhibition, regression, or stasis of a disease or disorder, or alleviating, lessening, suppressing, inhibiting, reducing the severity of, eliminating or substantially eliminating, or ameliorating a symptom of the disease or disorder.

As used herein, “effective” when referring to an amount of laquinimod refers to the quantity of laquinimod that is sufficient to yield a desired therapeutic response. Efficacy can be measured by e.g., one or more of the patient's Q-motor assessment, Unified Huntington's Disease Rating Scale (UHDRS) (Total Motor Score (TMS), functional capacity (TFC), Total functional assessment (FA) scale), MRI measure (of whole brain volume, caudate volume, white matter volume and ventricular volume), cognitive capacity in patients (e.g., cognitive assessment battery (HD-CAB) comprised of Symbol Digit Modalities Test (SDMT), Emotion Recognition, trail Making Test, Hopkins Verbal Learning Test, revised (HVLT-R) Pace Tapping at 3 Hz, One Touch Stocking of Cambridge (OTS, abbreviated 10 trial version), functional impairment due to cognitive decline (measured by Clinical Dementia Rating score Sum of Boxes (CRD-SB)), Physical Performance Test (PPT), Problem Based Assessment scale (PBA) short version, Hospital Anxiety and Depression Scale (HADS), Clinician's Interview-based Impression of Change plus Caregiver Input (CIBIC-Plus) global score, patient's quality of life as measured by Huntington's Disease Quality of Life (HD-QoL) and EQ5D instruments, the patient's work productivity, and reduction in brain atrophy as measured by change in whole brain volume, caudate volume and putamen volume.

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration. As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times weekly and so on, etc.

As used herein, “delay(ing) disease progression” in a subject afflicted with Huntington's disease means increasing the time to appearance of a symptom of Huntington's disease or a mark associated with Huntington's disease, or slowing the increase in severity of a symptom of Huntington's disease. For example, “delaying disease progression” in a subject afflicted with Huntington's disease can mean increasing the time until the subject reaches a certain UHDRS score. Further, “delay(ing) disease progression” as used herein includes reversing or inhibition of disease progression. “Inhibition” of disease progression or disease complication in a subject means preventing or reducing the disease progression and/or disease complication in the subject.

A “symptom” associated with Huntington's disease includes any clinical or laboratory manifestation associated with Huntington's disease and is not limited to what the subject can feel or observe.

As used herein, a subject “afflicted” with Huntington's disease means the subject has been diagnosed with Huntington's disease. In an embodiment, the patient is diagnosed with HD if the patient is determined to carry the mutated htt allele and shows motor symptoms above 5 points as measured on the UHDRS TMS scale.

As used herein, a subject at “baseline” is as subject prior to administration of laquinimod in a therapy as described herein.

As used herein, a subject who is “naïve” to a particular therapy is a subject who has not previously received said therapy.

A “pharmaceutically acceptable salt” of laquinimod as used in this application includes lithium, sodium, potassium, magnesium, calcium, manganese, copper, zinc, aluminum and iron. Salt formulations of laquinimod and the process for preparing the same are described, e.g., in U.S. Patent Application Publication No. 2005/0192315 and PCT International Application Publication No. WO 2005/074899, which are hereby incorporated by reference into this application.

Laquinimod can be administered alone but is generally mixed with suitable pharmaceutical diluents, extenders, excipients, or carriers (i.e., “pharmaceutically acceptable carriers”) suitably selected with respect to the intended form of administration and as consistent with conventional pharmaceutical practices. For example, laquinimod can be co-administered with the pharmaceutically acceptable carrier in the form of a tablet or capsule, liposome, or as an agglomerated powder. A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

A dosage unit may comprise a single compound or mixtures of compounds thereof. A dosage unit can be prepared for oral dosage forms, such as tablets, capsules, pills, powders, and granules.

Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Patent Application Publication No. 2005/0192315, PCT International Application Publication Nos. WO 2005/074899, WO 2007/047863, and 2007/146248. These references in their entireties are hereby incorporated by reference into this application.

General techniques and compositions for making dosage forms useful in the present invention are described—in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

It is understood that where a parameter range is provided, all integers within that range, and tenths thereof, are also provided by the invention. For example, “0.5-1.5 mg” includes 0.5 mg, 0.6 mg, 0.7 mg, etc. up to 1.5 mg.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1: Clinical Trial (Phase II)—Laquinimod for Treating Patients Afflicted with Huntington'Disease (HD)

A phase II, multi-centered, multinational, randomized, parallel-group, double-blinded, placebo-controlled study is conducted to evaluate the safety and efficacy of laquinimod (0.5, 1.0 and 1.5 mg/day) versus placebo in patients with HD.

### Laquinimod (LAQ)

Laquinimod is an immunomodulator under development for Multiple Sclerosis (MS), Crohn's Disease (CD), and Systemic Lupus Erythematosus (SLE). Studies investigating the mode of action of laquinimod have shown that its effect is possibly mediated by interference with the NF-kB pathway resulting In immunomodulation, including modulation of the cytokine balance and reduction of inflammation. Laquinimod is not a general immunosupressor, nor immunotoxic, but treatment instead results in a shift in the cytokine balance towards reduced pro-inflammatory cytokines, induction of regulatory monocytes, reduced astrogliosis, and reduced infiltration to inflammatory target tissues, as demonstrated in animal models of MB and CD.

### Huntington's Disease (HD)

HD is a hereditary disorder causing degeneration of neurons in the brain leading to uncontrolled movements, progressive loss of controlled motor function, cognitive decline, and emotional disturbance. The onset and progression varies but the most common age of onset is between 30 and 40 years. The illness is fatal and generally lasts 15-20 years.

A number of medications are used off-label to control motor and emotional problems arising from HD. The scientific evidence for these drugs in HD is poor and most of these drugs have significant side effects. None of the drugs used today has proven effect on disease progression.

It is believed that inflammatory process in the CNS contributes to the pathogenesis of HD, via neuronal disturbances and cell death. Microglia, the major intrinsic immunocompetent cells in the CNS are normally present in a quiescent state. Open exposure to neuronal insults such as infection, ischaemia or the presence of abnormal protein aggregations (including mutant huntingtin aggregation), microglia become activated and release pro-inflammatory cytokines and cytotoxic mediators. This may eventually contribute to neuronal death. Microglia activation was evident post mortom in HD patients (Sapp et al., 2001) as well as in-vivo in pre-symptomatic and symptomatic HD gene carriers, demonstrated by PET tracer ligands to activation markers on microglia (Tai Y F et al., 2007). In vivo microglia activation was in correlation with striatal neuronal dysfunction. These findings indicate that microglial activation is an early event in the pathogenic processes of HD and is associated with subclinical progression of disease. Elevated levels of inflammatory cytokines have been detected both in serum and cerebral spinal fluid in patients with HD. Specifically Interleukin (IL)-6 levels were increased in the plasma of pre-manifest HD gene carriers. In addition, monocytes from HD subjects as well as macrophages and microglia from the YAC128 HD model, were hyperactive in response to stimulation. Moreover, in a postmortem analysis of HD patients' striatum, RNA Levels of IL-6, IL-8, and TNF-α were significantly increased (Bjorkqvist et al, 2008). IL-6 release is triggered by activation of the NF-KB pathway. The increased cytokine release, in particular IL-6, correlates with the interesting finding that NF-KB activity is up-regulated in several HD cell models and transgenic mouse models, possibly by direct interaction of mutant htt and IKK (Khoshnan et al., 2004)

In Human HD studies, astrocytosis is observed in affected regions of the brain of patients with HD. The huntingtin protein co-localizes with these reactive astrocytes in specific regions (S. K. Singhrao et al 1998). Astrocytes from HD mice has been shown to have an aberrant activation of NF-κB and peripheral monocytes from HD patients express a hyper-reactive phenotype. The data collected to date suggests that laquinimod may (i) reduce the levels of proinflammatory cytokines such as TNFα; (ii) reduce inflammation within the CNS; (iii) down-regulate genes involved in inflammation and antigen presentation; and (iv) modulate T-cell responses via a direct effect on antigen presenting cells, and skew monocytes to a regulatory phenotype. The presumed mechanism by which laquinimod exerts this effect is down-regulation of both astrocytic and microglial pro-inflammatory response mediated by interference with the NF-κB pathway, investigated in experimental autoimmune encephalomyelitis (EAE) and the Cuprizone models of demyelination (Wegner, 2010; Bruck, 2012; Aharoni, 2012).

No clinical data on the effects of laquinimod in patients with HD is previously reported. However, clinical data from patients with relapsing remitting MS show a benefit of laquinimod treatment on brain atrophy and disability progression after 1 year of treatment, also in patients without relapses during this period. A disproportionally large effect on disability compared to relapses was also observed. The results suggest that in addition to inflammatory modulating effects, laquinimod also has neuroprotective effects, and a mode of affecting CNS inflammatory processes beyond the classical MS dogma of active T cell driven lesions.

In humans, laquinimod is extensively metabolized by CYP3A4 in the liver, and its PK is affected by moderate and strong CYP3A4 inhibitors, strong CYP3A4 inducers, and moderate hepatic impairment. Clinical pharmacology studies show that laquinimod has a predictable and linear PK profile with high plasma binding high plasma protein binding (>98%), high oral bioavailability (˜90%), low oral clearance (˜0.09 L/h), low apparent volume of distribution (˜10 L) and long half-life (˜80 h).

HD manifests in 3 domains; motor, cognition, psychiatric, (function), all assessed by various rating scales, whereof none has been formally validated in a regulatory perspective.

This is the first clinical study with laquinimod in HD, and the mode of action of laquinimod does not speak for a benefit in a given domain of the disease. In addition, no validated biomarker proven to correlate with clinical benefit from drug intervention is available (as no effective drugs are available), but whole brain volume and caudate volume measured by MRI have been reported to correlate with clinical progression in longitudinal studies.

### Summary

This study includes 4 treatment arms, with approximately 100 patients per treatment arm and approximately 400 patients in total. The study is conducted in approximately 30 centers in Canada, USA and Europe.

### Study Population

The study population is comprised of patients with adult onset HD, with a cytosine-adenosine-guanine (CAG) repeat length between 40 and 49, inclusive. The basic eligibility criteria selects a population with symptoms of HD, as assessed by a Unified HD Rating Scale−Total Motor Score (UHDRS−TMS)>5, but with a largely retained functional capacity, as assessed with a Unified HD Rating Scale−Total Functional Capacity (UHDRS−TFC) score≧8.

### Primary Study Objective

The primary objective of this study is to assess the efficacy of laquinimod 0.5, 1.0, and 1.5 mg qd in patients with HD after 12 months of treatment using the UHDRS−TMS.

### Secondary Study Objective

1. To assess the effect of laquinimod on brain atrophy in patients with HD after 12 months of treatment using MRI measures of caudate volume.
2. To assess the effect of laquinimod on the cognitive capacity in patients with HD after 12 months of treatment using the cognitive assessment battery (CAB) for patients with HD [comprised of: Symbol Digit Modalities Test (SDMT), Emotion Recognition, Trail Making Test, Hopkins Verbal Learning Test, revised (HVLT-R), Paced Tapping at 3 Hz, One Touch Stockings of Cambridge (OTS, abbreviated 10 trial version)].
3. To assess the effect of laquinimod on the clinical global impression in patients with HD after 12 months of treatment using CIBIC-Plus.
4. To assess the effect of laquinimod on the functional capacity in patients with HD after 12 months of treatment using the UHDRS−TFC scale.

### Exploratory Study Objective

1. To assess the effect of laquinimod on brain atrophy in patients with HD after 12 months of treatment using MRI measures of whole brain volume, caudate volume, white matter volume, and ventricular volume.
2. To assess the effect of laquinimod on the functional capacity in patients with HD after 12 months of treatment using the UHDRS-Functional Assessment (FA) scale.
3. To assess the effect of laquinimod on motor function in patients with HD after 12 months of treatment using the objective instrument Q-Motor.
4. To assess the effect of laquinimod on physical performance in patients with HD after 12 months of treatment using the modified Physical Performance Test (mPPT).
5. To assess the effect of laquinimod on quality of life in patients with HD after 12 months of treatment using the HD Quality of Life (HD-QoL) and EQ5D instruments.
6. To assess the effect of laquinimod on work productivity in patients with HD after 12 months of treatment.
7. To assess the effect of laquinimod on functional impairment due to cognitive decline in patients with HD after 12 months of treatment using the Clinical Dementia Rating score Sum of Boxes (CDR-SB).
8. To assess the effect of laquinimod on depression and anxiety in patients with HD after 12 months of treatment using the Hospital Anxiety and Depression Scale (HADS).
9. To assess the effect of laquinimod on behavioral signs and symptoms in patients with HD after 12 months of treatment using the Problem Based Assessment scale, short form (PBA-s).
10. To evaluate the pharmacokinetics of laquinimod in patients with HD.
11. To investigate the relationship between exposure to laquinimod and outcome measures (e.g., clinical effect and toxicity parameters).

### Ancillary Objectives (Sub Studies)

1. Exploration of correlation between genetic polymorphisms in DNA and pharmacokinetics, clinical response to laquinimod, and/or adverse drug reactions.
2. Exploration of correlation between RNA expression profile in blood cells and clinical response to laquinimod.
3. Exploration of changes in blood cell's gene expression profile as potential biomarkers for laquinimod mechanism of action.
4. Evaluation of changes in cytokines and other soluble protein levels as potential biomarkers for laquinimod mechanism of action and/or response predictive factors.
5. Exploration of gene expression and/or protein profile in monocytes in response to laquinimod treatment.
6. Exploration of change in microglial activation state in response to treatment with laquinimod.
7. Exploration of effect on metabolic changes in the putamen and frontal white matter that are associated with the earliest stages of HD.

### Investigational Medicinal Product (IMP) & Dosage

The dose levels of laquinimod are 0.5 mg/day, 1.0 mg/day and 1.5 mg/day. Every patient takes 3 capsules once daily at the same time of the day for the whole study period.

The Laqunimod Treatment Arms are as follows:
1.5 mg LAQ/day: patients randomized to laquinimod 1.5 mg qd (i.e., once daily) treatment arm receive 3 capsules of 0.5 mg laquinimod daily.
1.0 mg LAQ/day: patients randomized to laquinimod 1.0 mg qd treatment arm receive 2 capsules of 0.5 mg laquinimod and 1 capsule of matching placebo daily.
0.5 mg LAQ/day: patients randomized to laquinimod 0.5 mg qd treatment arm receive 1 capsule of 0.5 mg laquinimod and 2 capsules of matching placebo daily.

In addition, the Placebo Arm is as follows:
Placebo: Patients randomized to the placebo treatment arm receive 3 capsules of matching placebo capsules daily.

The 0.5 mg laquinimod capsules were prepared using 0.534 mg of laquinimod sodium per capsule (which is equivalent to 0.5 mg of laquinimod acid). The capsules were prepared using a blend proportional to the 0.6 mg capsules described in PCT International Application No. PCT/US2007/013721 (WO 2007/146248). The capsules were prepared according to the method described in PCT International Application No. PCT/US2007/013721 (WO 2007/146248), which is hereby incorporated by reference into this application.

Randomization is performed by IRT using dynamic randomization to balance the treatment groups within centers. Subjects are equally assigned to the 4 treatment groups (3 active treatment groups and placebo, with allocation ratio of 1:1:1:1).

### Study Duration

Total study participation is up to 14 months:

Screening: 2-5 weeks

Treatment period: 12 months double-blind, placebo-controlled treatment

Safety Follow-up period: 1 month safety follow-up period following the last dose of study medication.

### Study Design

This is a multinational, multicenter, randomized, double-blind, parallel-group, placebo-controlled study to evaluate the safety and clinical effect of daily oral administration of laquinimod (0.5 mg, 1.0 mg, or 1.5 mg) in patients with HD.

Patients are treated with laquinimod for 12 months, and safety and efficacy are assessed after 1, 3, 6, 9 and 12 months of treatment. Eligible subjects are randomized in a 1:1:1:1 ratio into one of the following treatment arms:
1. Laquinimod capsule 0.5 mg (Total of 0.5 mg)
2. Laquinimod capsules 0.5 mg×2 (Total of 1.0 mg)
3. Laquinimod capsules 0.5 mg×3 (Total of 1.5 mg)
4. Matching placebo

The following assessments are performed at the specified time points:
1. Eligibility criteria is reviewed and confirmed at screening and baseline.
2. Vital signs are measured at each study visit.
3. A physical examination is performed at each study visit.
4. The following safety clinical laboratory tests are performed:
   a) Complete blood count (CBC) with differential at each study visit.
   b) Serum chemistry (including electrolytes, liver enzymes, urea, creatinine, glucose, total protein, albumin, direct and total bilirubin, Creatinephosphokinase (CPK), serum conventional C-reactive protein (CRP), fibrinogen and pancreatic amylase)—at all scheduled visits. Calculated Glomerular Filtration Rate (GFR) is assessed at screening and prior to each MRI scan.
   c) Lipid profile (total cholesterol, HDL, LDL, triglycerides)—at baseline (month 0) and 12 months.
   d) Serum TSH, T3 and Free T4 at baseline (month 0), month 6 and month 12.
   e) Urinalysis at the screening visit.
   f) Serum human choriogonadotropin beta (β-hCG) in women of child-bearing potential is performed at each scheduled study visit.
   g) Urine β-hCG test is performed in women of child-bearing potential at baseline (month 0) and at each scheduled study visit thereafter.
   h) Starting after visit Month 3 a urine β-hCG test is performed in women of child-bearing potential every 28 (±2) days. In case of suspected pregnancy (positive urine β-hCG test result), the subject is instructed to return within 10 days with all remaining study drugs capsules.
5. Additional 10 mL of blood for analysis of protein serum levels via the Rules-Based Medicine biomarker discovery platform or similar is collected at baseline, and months 6 and 12, concomitant with other blood draw procedures.
6. ECG is performed at screening and baseline, and at month 1, 3, 6, and 12.
7. 24-h ECG profiling is collected for concentration/effect modeling at selected sites at month 6 from in total 75 patients (15 per dose).
8. Chest X-ray is performed at screening (if not performed within 6 months prior to the screening visit).
9. Blood sample for genomic analysis and CAG repeat length determination is drawn at screening.
10. Adverse Events (AEs) are monitored throughout the study.
11. Suicidality is monitored throughout the study through administration of the C-SSRS.
12. Concomitant Medications are monitored throughout the study.
13. MRI scans at baseline and month 12 for all subjects.
14. Motor function evaluations (UHDRS Total Motor Score, and Q motor) is performed at screening, baseline and at months 3, 6, 9, and 12.
15. Global functional capacity evaluations (Physical Performance test (PPT), UHDRS−Total Functional Capacity, and CIBIC-plus) is performed baseline and at months 6 and 12.
16. Psychiatric and behavioral evaluations (PBA-s, and HADS) at baseline and at months 6 and 12.
17. Cognitive capacity is evaluated at screening, baseline and at months 6 and 12, by administration of the CAB for HD (Symbol Digit Modalities Test (SDMT), Emotion Recognition, Trail Making Test, Hopkins Verbal Learning Test, revised (HVLT-R), Paced Tapping at 3 Hz, One Touch Stockings of Cambridge (OTS, abbreviated 10 trial version).
18. Cognitive functional capacity is assessed at baseline and at months 6 and 12, by clinician rating of the CDR-SB scale including information from the patient and the informant, and the sum of boxes score is calculated.
19. Quality of life is assessed by the HD-QoL questionnaire at baseline and month 12.
20. Pharmacokinetic (PR) study: Blood samples for analysis of laquinimod plasma concentrations is collected from all subjects at months 1, 6 and 9.
21. Blood is collected for 24-h PK profiling at selected sites at month 6 from in total 75 patients (15 per dose).

For patients participating in the ancillary studies:
1. Blood is collected for 24-h pharmacokinetic (PK) profiling at selected sites at Month 1 from in total 60 patients (15 per treatment group).
2. Blood for monocyte gene expression and protein profile is collected in a subgroup of patients at baseline and Month 12.
3. PET scan at selected sites in a subgroup of patients at baseline and Month 122.
4. MRI scans for MRS evaluation is done in a subgroup of patients at baseline and Month 12.

### Inclusion/Exclusion Criteria

### Inclusion Criteria

Subjects must meet all the inclusion criteria to be eligible:
1. Presence of 40-49CAG repeats, inclusive, in the huntingtin gene based on centralized CAG testing during screening.
2. Male or female between 21-55 years of age, inclusive, with an onset of HD at or after 18 years of age.
3. Females of child bearing potential (women who are not post-menopausal or have undergone surgical sterilization) must practice an acceptable method of birth control for 30 days before the study treatment, 2 acceptable methods of birth control throughout the duration of the study, until 30 days after the last dose of treatment is taken. Acceptable methods of birth control in this study include: Intrauterine devices, barrier methods (condom or diaphragm with spermicide) and hormonal methods of birth control (e.g., oral contraceptive, contraceptive patch, long-acting injectable contraceptive).
4. Male patients whose partner is pregnant or of child-bearing potential and not using effective contraception must use a condom (with spermicide if available) throughout treatment duration and until 30 days after the last dose of treatment is administered.
5. A sum of >5 points on the UHDRS−TMS at the screening visit
6. UHDRS−TFC≧8 at the screening visit.
7. Able and willing to provide written informed consent prior to any study related procedure being performed at the screening visit. Patients with a legal guardian should be consented according to local requirements.
8. Willing to provide a blood sample for CAG analysis at the screening visit.
9. Willing and able to take oral medication and able to comply with the study specific procedures.
10. Ambulatory, being able to travel to the study centre, and likely to be able to continue to travel for the duration of the study.
11. Availability and willingness of a caregiver, informant, or family member to provide input at study visits assessing CIBIC-Plus, CDR-SB, PBA-s, and HD-QoL. A caregiver is recommended to be someone who attends to the patient at least 2 to 3 times per weeks for at least 3 hours per occasion.
12. For patients taking allowed antidepressant medication, the dosing of medication must have been kept constant for at least 30 days before baseline and must be kept constant during the study.

### Exclusion Criteria

Any of the following excludes the subject from entering the study:
1. Use of immunosuppressive agents, or cytotoxic agents, including cyclophosphamide and azatioprine within 12 months prior to screening.
2. Previous use of laquinimod.
3. Use of moderate/strong inhibitors of cytochrome P450 (CYP)3A4 within 2 weeks prior to randomization.
4. Use of inducers of CYP3A4 within 2 weeks prior to randomization.
5. Pregnant or breastfeeding.
6. Serum levels≧3×upper limit of the normal range (ULN) of either alanine aminotransferase (ALT) or aspartate aminotransferase (AST) at screening.
7. Serum direct bilirubin which is ≧2×ULN at screening.
8. Creatinine clearance <60 mL/min at screening, calculated using the Cockcroft Gault equation: (140 age)×mass (kg)×[0.85 if female]/72×serum creatinine (mg/dL)×88.4.
9. Subjects with a clinically significant or unstable medical or surgical condition that may put the patient at risk when participating in the study or may influence the results of the study or affect the patient's ability to take part in the study, as determined by medical history, physical examinations, ECG, or laboratory tests. Such conditions may include:
   a) A major cardiovascular event (e.g. myocardial infarction, acute coronary syndrome, de-compensated congestive heart failure, pulmonary embolism, coronary revascularization) that occurred during the past 6 months prior to randomization.
   b) Any acute pulmonary disorder
   c) A central nervous system (CNS) disorder other than HD that may jeopardize the subject's participation in the study, including such disorders that are demonstrated on the baseline magnetic resonance imaging (MRI) (based on local read).
   d) A gastrointestinal disorder that may affect the absorption of study medication.
   e) Renal disease.
   f) Cirrhotic patients with moderate or severe hepatic impairment
   g) Known human immunodeficiency virus (HIV) positive status. Patients undergo an HIV test at screening per local requirements, if applicable.
   h) Any malignancies, excluding basal cell carcinoma, in the 5 years prior to randomization.
10. Any clinically significant, abnormal, screening laboratory result which affects the patients' suitability for the study or puts the patient at risk if he/she enters the study.
11. Unsuitable for MRI (e.g, claustrophobia, metal implants).
12. Alcohol and/or drug abuse within the 6 months prior to screening, as defined by Diagnostic and Statistical Manual of Mental Disorders—Fourth Edition Text Revision (DSM-IV TR) criteria for substance abuse.
13. Patients with active suicidal ideation as measured by a most severe suicide ideation score of 4 (Active Suicidal Ideation with Some Intent to Act, without Specific Plan) or 5 (Active Suicidal Ideation with Specific Plan and Intent) on the Columbia-Suicide Severity Rating Scale (C-SSRS) or subjects who answer “Yes” on any of the 5 C-SSRS Suicidal Behavior Items (actual attempt, interrupted attempt, aborted attempt, preparatory acts, or behavior) or subjects who present a serious risk of suicide.
14. Patients with known intracranial neoplasms, vascular malformations, or intracranial hemorrhage.
15. Known drug hypersensitivity that would preclude administration of laquinimod or placebo, such as hypersensitivity to mannitol, meglumine or sodium stearyl fumarate.
16. Swallowing difficulties that would preclude administration of laquinimod or placebo capsules.
17. Treatment with any investigational product within 12 weeks of screening or patients planning to participate in another clinical study assessing any investigational product during the study. Patients in non-interventional and/or observational studies are excluded from this study.
18. Treatment with tetrabenazine within 30 days of the study baseline visit.
19. Treatment with antipsychotic medication within 30 days of the study baseline visit.

### Outcome Measures

### Primary Efficacy Variable and Endpoint

The primary efficacy variable and endpoint for this study is change from baseline in the UHDRS−TMS (defined as the sum of the scores of all UHDRS−TMS subitems) at Month 12/Early Termination (ET) (evaluated at baseline and Months 1, 3, 6 and 12).

### Secondary Efficacy Variable and Endpoint

1. Percent change from baseline in caudate volume at Month 12/ET (evaluated at baseline and Month 12).
2. Change from baseline in HD-CAB total score (sum of the standardized sub-components at Month 12/ET (evaluated at baseline and Months 6 and 12).
3. CIBIC-Plus global score at Month 12/ET (evaluated at Months 6 and 12) as compared to baseline (rated by an independent rater).
4. Change from baseline in UHDRS−TFC at Month 12/ET (evaluated at baseline, Months 6 and 12).

### Exploratory Efficacy Variables and Endpoints

1. Change from baseline in brain atrophy as defined by the percentage change in volume in: whole brain volume, caudate volume and white matter volume at Month 12/ET and the absolute change in ventricular volume at month 12/ET (evaluated at baseline and Month 12).
2. Change from baseline in UHDRS−FA at Month 12/ET (evaluated at baseline and Month 6 and Month 12).
3. Change from baseline in Q-Motor assessment at Month 12/EDT (evaluated at baseline and Months 1, 3, 6 and 12).
4. Change from baseline in modified Physical Performance Test (mPPT) at Month 12/ET (evaluated at baseline and Month 6 and 12).
5. Change from baseline in HD-QoL and EQ5D at Month 12/ET (evaluated at baseline and Month 12).
6. Change from baseline in WLQ at Month 12/ET (evaluated at baseline and Month 12).
7. Change from baseline in cognitive assessment battery (HD-CAB) at Month 12/ET (evaluated at baseline and Months 6 and 12): (Symbol Digit Modalities Test (SDMT), Emotion Recognition, Trial Making Test, Hopkins Making Test, Hopkins Verbal Learning Test, revised (HVLT-R); Paced Tapping at 3 Hz, One Touch Stockings of Cambridge (OTS, abbreviated 10 trial version)).
8. Change from baseline in CDR-SB at Month 12/ET (evaluated at baseline and Months 6 and 12).
9. Change from baseline in PBA-short (PBA-s) at Month 12/ET (evaluated at baseline and Months 1, 3, 6 and 12).
10. Change from baseline in HADS at Month 12/ET (evaluated at baseline and Months 1, 3, 6 and 12).

### Safety/Tolerability

Safety variables and endpoints include the following:
1. Adverse Events reports throughout the study.
2. ECG findings throughout the study.
3. Clinical safety laboratory throughout the study.
4. Vital signs measurements throughput the study.
5. Physical examination findings throughout the study.
6. Changes from baseline suicidality (C-SSRS) throughout the study.
7. Proportion of subjects (%) who prematurely discontinued from the study, reason of discontinuation and the time to ET.
8. Proportion of subjects (%) who prematurely discontinued from the study due to AEs and the time to ET.

### Pharmacokinetics/Pharmacodynamics

Pharmacogenomic (PGx) assessment includes DNA variations and RNA, gene expression pattern associated with clinical treatment responses to laquinimod (e.g. clinical effect, Q-Motor, pharmacokinetics, tolerability, and safety features or disease susceptibility and severity features). Samples for DNA analysis are collected at screening (or if not possible, at the next possible visit). Samples for RNA analysis are collected at baseline, Month 6 and 12.

### Ancillary Studies

1. Microglial activation state is investigated at selected sites and patients (N˜20/treatment arm). Scans and imaging analysis of microglial activation marker translocator protein (TSPO) is performed at baseline and Month 12.
2. Change in putaminal and frontal white matter markers of neuronal integrity (NAA) and astrocytosis (myoinositol) is investigated at selected sites using MRS (N˜20/treatment arm) at baseline and Month 12.
3. Monocyte gene expression and/or protein profile in response to treatment with laquinimod is analyzed at selected sites and patients (N˜20/treatment arm). Monocytes are separated from isolated peripheral blood mononuclear cells (PBMC) and analyzed for gene expression and/or protein profile at baseline and Month 12.
4. Peripheral cytokine and proteomic analysis in response to treatment with laquinimod are investigated in a subgroup of patients at selected sites at baseline and Months 6 and 12.

### Statistical Considerations

### Sample Size

This study aims to detect beneficial effects in deteriorating clinical signs and symptoms. Based on previous studies in patients with HD, the UHDRS−TMS has been shown to be one of the more sensitive clinical measures to detect decline in symptoms of HD. It is estimated that approximately 100 patients per arm enables a power of 80% to detect a beneficial effect of 2.5 points or more in the change from baseline in UHDRS−TMS of an active laquinimod arm compared to placebo, assuming SD of 6.2 and type I error of 5%.

As the intention is to investigate laquinimod as a treatment to slow disease progression and prohibit neuronal death in the CNS, the study is sized to detect changes in brain atrophy rate after treatment. One of the most sensitive measures to detect brain atrophy over time in patients with HD is change in the caudate volume. Approximately 100 patients per arm enables a power of 80% to detect a beneficial effect of 0.95 (30% of the estimated decline in placebo) or more in the percent change from baseline in caudate brain atrophy of an active laquinimod arm compared to placebo, assuming SD of 2.36 and type I error of 5%.

### Primary Efficacy Endpoints Analyses

The change from baseline UHDRS−TMS is analyzed using a Repeated Measures model (SAS® MIXED procedure with REPEATED sub-command). The model includes the following fixed effects: categorical week in trial by treatment interaction, center, and UHDRS−TMS at baseline. The analysis uses unstructured covariance matrix for repeated observations within patients. If the model does not converge, the Maximum-Likelihood (ML) estimation method is used instead of the default Restricted ML (REML). If the model still does not converge then a simpler covariance structures with less parameters is used, according to the following order: Heterogeneous Autoregressive (1) [ARH(1)], Heterogeneous Compound Symmetry (CSH), Autoregressive (1) [AR(1)], and Compound Symmetry (CS). The estimated means at the Month 12 visit is compared between the active treatment arms and the placebo arm.

### Secondary Efficacy Endpoints Analyses

According to the hierarchical method to control inflation in type I error rate for multiple endpoints, any statistically significant dose observed in the primary analysis continues to be tested for the secondary endpoints at an alpha level of 5%, according to the secondary endpoints order.

The secondary efficacy endpoints: change from baseline in HD-CAB total score and change from baseline in UHDRS−TFC, is analyzed in the same way as the primary efficacy endpoint except that the efficacy endpoint evaluation at baseline is included in the model instead of baseline UHDRS−TMS.

CIBIC-Plus is analyzed in the same way as described above except that the baseline Clinician's Interview-based Impression of severity (CIBIS) is included in the model as the efficacy measure at baseline.

The percent change from baseline to Month 12/ET in caudate volume is analyzed using an Analysis Of Covariance (ANCOVA) model (SAS® MIXED procedure). The model includes the following fixed effects: treatment, center, and caudate volume at baseline. The estimated means at the Month 12 visit is compared between the active treatment arms and the placebo arm. Early terminated patient observation have their Last Observation Carried Forward (LOCF).

### Results

0.5 mg/day, 1.0 mg/day and 1.5 mg/day oral dose of laquinimod is effective to treat symptomatic early HD patients (Unified HD Rating Scale (UHDRS)−Total Motor Score (TMS) of >5 and/or Unified HD Rating Scale (UHDRS)−Total Functional Capacity (TFC) of ≧8 at baseline). 0.5 mg/day, 1.0 mg/day and 1.5 mg/day oral dose of laquinimod also delay disease progression in symptomatic early HD patients in that:
1. Progression (rate of change) of UHDRS−TMS (defined as the sum of all UHDRS motor domains ratings) is slower in patients in the Laquinimod Treatment Arm as compared to control subjects (patients in the Placebo Arm).
2. Progression (rate of change) of brain atrophy (as defined by the percentage change in volume in Whole brain volume, Caudate volume, white matter volume, and ventricular volume) is slower in patients in the Laquinimod Treatment Arm as compared to control subjects (patients in the Placebo Arm).
3. Progression (rate of change) of Q-motor assessments score is slower in patients in the Laquinimod Treatment Arm as compared to control subjects (patients in the Placebo Arm).
4. Progression (rate of change) deterioration of functional capacity using of UHDRS−Total functional capacity (TFC) score is slower in patients in the Laquinimod Treatment Arm as compared to control subjects (patients in the Placebo Arm).
5. Progression (rate of change) of UHDRS−FA score is slower in patients in the Laquinimod Treatment Arm as compared to control subjects (patients in the Placebo Arm).
6. Progression (rate of change) of cognitive assessment battery (CAB) (Symbol Digit Modalities Test (SDMT), Emotion Recognition, Trail Making Test, Hopkins Verbal Learning Test, revised (HVLT-R), Paced Tapping at 3 Hz, One Touch Stockings of Cambridge (OTS, abbreviated 10 trail version) is slower in patients in the Laquinimod Treatment Arm as compared to control subjects (patients in the Placebo Arm).
7. Progression (rate of change) of Physical Performance Test (PPT) score is slower in patients in the Laquinimod Treatment Arm as compared to control subjects (patients in the Placebo Arm).
8. Progression (rate of change) of PBA (short) is slower in patients in the Laquinimod Treatment Arm as compared to control subjects (patients in the Placebo Arm).
9. Progression (rate of change) of HADS is slower in patients in the Laquinimod Treatment Arm as compared to control subjects (patients in the Placebo Arm).
10. Progression (rate of change) of CIBIC-Plus global score is slower in patients in the Laquinimod Treatment Arm as compared to control subjects (patients in the Placebo Arm).
11. Progression (rate of change) of the patient's work productivity and quality of life (measured by HD-QoL) is slower in patients in the Laquinimod Treatment Arm as compared to control subjects (patients in the Placebo Arm).

### REFERENCES

1. Aharoni et al. (2012) “Oral treatment with laquinimod augments regulatory T-cells and brain-derived neurotrophic factor expression and reduces injury in the CNS of mice with experimental autoimmune encephalomyelitis.” J Neuroimmunol. 2012 Oct. 15; 251(1-2):14-24.
2. Bechtel et al. (2010) “Tapping linked to function and structure in premanifest and symptomatic Huntington disease.” Neurology. 2010 Dec. 14; 75(24):2150-60.
3. Bjorkqvist et al. (2008) “A novel pathogentic pathway of immune activation detectable before clinical onset in Huntington's disease” J. Exp Med. 2008 Aug. 4; (2058):1869-77.
4. Bowie and Harvey (2006) “Administration and interpretation of the Trail Making Test.” Nat Protoc. 2006; 1(5):2277-81.
5. Boyes et al. (2007) “Intensity non-uniformity correction using N3 on 3T scanners with multichannel phased array receiver coils.” NeuroImage 2007, doi:10.1016/j. neuroima 2007 Oct. 26
6. Brown et al. (2000) “Physical and performance measures for the identification of mild to moderate frailty.” J Gerontol A Biol Sci Med Sci. 2000 June; 55(6):M350-5.
7. Brück et al. (2012) “Reduced astrocytic NF-κB activation by laquinimod protects from cuprizone-induced demyelination.” Acta Neuropathol. 2012 September; 124(3):411-24.
8. Brück et al., (2011) “Insight into the mechanism of laquinimod action”, J. Neurol Sci. 306:173-179.
9. Ciammola et al. (2007) “Low brain-derived neurotrophic factor (BDNF) levels in serum of Huntington's disease patients”. Am J Med Gent Part B, 144b:574-577.
10. Craufurd et al. (2001) “Behavioral changes in Huntington Disease.” Neuropsychiatry Neuropsychol Behav Neurol. 2001 October-December; 14(4):219-26.
11. De Souza et al. (2010) “Validation of self-report depression rating scales in Huntington's disease.” Mov Disord. 2010 Jan. 15; 25(1):91-6.
12. EuroQol Group. EuroQol-a new facility for the measurement of health-related quality of life. Health Policy 1990; 16:199-208.
13. Freeborough and Fox (1997) “The boundary shift integral: an accurate and robust measure of cerebral volume changes from registered repeat MRI.” IEEE Trans Med Imaging 1997; 16:623-629.
14. Freeborough et al. (1997) “Interactive algorithms for the segmentation and quantitation of 3-D MRI brain scans.” Comput Methods Programs Biomed 1997; 53:15-25.
15. Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. For Biologics Eval. and Res., Clin./Pharm., November 1999 <http://www.fda.gov/cber/gdlns/metabol.pdf>.
16. Gurevich et al. (2010) “Laquinimod suppress antigen presentation in relapsing-remitting multiple sclerosis: invitro high-throughput gene expression study.” J Neuroimmunol, 2010 Apr. 15; 221(1-2):87-94.
17. Hobbs et al. (2009) “Automated quantification of caudate atrophy by local registration of serial MRI: evaluation and application in Huntington's disease.” Neuroimage 2009; 47:1659-1665.
18. Hocaoglu et al. (2012) “The Huntington's Disease health-related Quality of Life questionnaire (HDQoL): a disease-specific measure of health-related quality of life.” Clin Genet. 2012 February; 81(2):117-22.
19. Huntington Study Group (1996) “Unified Huntington's Disease Rating Scale: Reliability and Consistency” Movement Disorders; 11(2):136-142.
20. Joffres et al. (2000) “Qualitative analysis of the clinician interview-based impression of change (Plus): methodological issues and implications for clinical research.” Int Psychogeriatr. 2000 September; 12(3):403-13.
21. Khoshnan at al. (2004) “Activation of the IkappaB kinase complex and nuclear factor-kappaB contributes to mutant huntingtin neurotoxicity.” J Neurosci. 2004 Sep. 15; 24(37):7999-8008.
22. Kingma et al. (2008) “Behavioural problems in Huntington's disease using the Problem Behaviours Assessment.” Gen Hosp Psychiatry. 2008 March-April; 30(2):155-61.
23. Lerner et al. (2001) “The Work Limitations Questionnaire.” Med Care. 2001 January; 39(1):72-85.
24. Leung et al. (2010) “Robust atrophy rate measurement in Alzheimer's disease using multi-site serial MRI: tissue-specific intensity normalization and parameter selection.” Neuroimage 2010; 50:516-523.
25. Morris J C. (1993) “The Clinical Dementia Rating (CDR): current version and scoring rules.” Neurology. 1993; 43(11):2412-2414.
26. O'Bryant et al (2008) “Texas Alzheimer's Research Consortium. Staging dementia using Clinical Dementia Rating Scale Sum of Boxes scores: a Texas Alzheimer's research consortium study.” Arch Neurol. 2008 August; 65(8):1091-5.
27. PCT International Application Publication No. WO 2007/0047863, published Apr. 26, 2007.
28. PCT International Application Publication No. WO 2007/0146248, published Dec. 21, 2007.
29. Politis et al. (2012) “Imaging of microglia in patients with neurodegenerative disorders.” Front Pharmacol. 2012 May 29; 3:96.
30. Polman et al. (2005) “Diagnostic criteria for multiple sclerosis: 2005 revisions to the McDonald Criteria.” Annals of Neurology, 58(6):840-846.
31. Polman et al. (2005) “Treatment with laquinimod reduces development of active MRI lesions in relapsing MS.” Neurology. 64:987-991.
32. Polman et al. (2011) “Diagnostic Criteria for Multiple Sclerosis: 2010 Revisions to the McDonald Criteria.” Ann Neural, 69:292-302.
33. Posner et al. (2011) “The Columbia-Suicide Severity Rating Scale: initial validity and internal consistency findings from three multisite studies with adolescents and adults.” Am J Psychiatry. 2011 December; 168(12):1266-77.
34. Reilmann et al. (2001) “Objective assessment of progression in Huntington's disease: a 3-year follow-up study.” Neurology. 2001 57(5):920-4.
35. Reilmann et al. (2009) “A teaching film, video library and online certification for the Unified Huntington's Disease Rating Scale Total Motor Score.” Aktuelle Neurologie; 2009; 3(S2):116
36. Reilmann et al. (2010) “Grasping premanifest Huntington's disease—shaping new endpoints for new trials. Mov Disord. 2010; 25(16):2858-62.
37. Reilmann et al. (2011) “Assessment of involuntary choreatic movements in Huntington's disease-toward objective and quantitative measures.” Mov Disord. 2011 October; 26(12):2267-73.
38. Reilmann R. (2012) “Huntington's disease: towards disease modification-gaps and bridges, facts and opinions.” Basal Ganglia, 2012; 2:241-248.
39. Rudick et al. (1999) “Use of the brain parenchymal fraction to measure whole brain atrophy in relapsing-remitting MS: Multiple Sclerosis Collaborative Research Group”. Neurology. 53:1698-1704.
40. Runström et al. (2002) “Laquinimod (ABR-215062) a candidate drug for treatment of Multiple Sclerosis inhibits the development of experimental autoimmune encephalomyelitis in IFN-β knock-out mice” (Abstract), Medicon Valley Academy, Malmoe, Sweden.
41. Sandberg-Wollheim et al. (2005) “48-week open safety study with high-dose oral laquinimod in patients.” Mult Scler. 11:S154 (Abstract).
42. Sapp et al. (2001) “Early and progressive accumulation of reactive microglia in the Huntington disease brain.” J Neuropathol Exp Neurol. 2001 February; 60(2):161-72.
43. Singhrao et al. (1998) “Huntingtin protein colocalizes with lesions of neurodegenerative diseases: An investigation in Huntington's, Alzheimer's, and Pick's diseases.” Exp Neurol. 1998 April; 150(2):213-22.
44. Sled et al. (1998) “A nonparametric method for automatic correction of intensity nonuniformity in MRI data.” IEEE Trans Med Imaging 1998; 17:87-97.
45. Sturrock et al. (2010) “Magnetic resonance spectroscopy biomarkers on pre-manifest and early Huntington's disease.” Neurology 2010, 75:1702-1710.
46. Tabrizi et al. (2012) “Potential endpoints for clinical trials in premanifest and early Huntington's disease in the TRACK-HD study: analysis of 24 month observational data.” Lancet Neurol 2012; 11:42-53.
47. Tabrizi et al. (2011) “Biological and clinical changes in premanifest and early stage Huntington's disease in the TRACK-HD study: the 12-month longitudinal analysis.” Lancet Neurol 2011; 10:31-42.
48. Tai et al. (2007) “Microglial activation in presymptomatic Huntington's disease gene carriers.” Brain. 2007 July; 130(Pt 7):1759-66.
49. U.S. Pat. No. 6,077,851, issued Jun. 20, 2000 (Bjork et al).
50. U.S. Pat. No. 7,589,208, issued Sep. 15, 2009 (Jansson et al).
51. United Nations Educational, Scientific and Cultural Organization (UNESCO), (2012) International Standard Classification of Education ISCED 2011, UNESCO Institute for Statistics, Montreal, Quebec, Canada.
52. Wegner et al. (2010) “Laquinimod interferes with migratory capacity of T cells and reduces IL-17 levels, inflammatory demyelination and acute axonal damage in mice with experimental autoimmune encephalomyelitis.” J Neuroimmunol. 2010 Oct. 8; 227(1-2):133-43.
53. Yang et al. (2004) “Laquinimod (ABR-215062) suppresses the development of experimental autoimmune encephalomyelitis, modulates the Th1/Th2 balance and induces the Th3 cytokine TGF-β in Lewis rats.” J. Neuoroimmunol. 156:3-9.
54. Zigmond and Snaith (1983) “The Hospital Anxiety and Depression Scale.” Acta Psychiatr Scand 1983; 67:361-370.
55. Fujita et al., 2008.
56. Imaizumi 2008.
57. Kreisl et al., 2010.
58. Kreisl et al., 2013.
59. Owen et al., 2011.
60. Owen et al., 2012.

What is claimed is:

1. A method of delaying disease progression in a subject afflicted with Huntington's disease comprising administering to the subject 0.5-1.5 mg/day of laquinimod thereby delaying disease progression in the subject, wherein laquinimod is preferably laquinimod sodium.

2. The method of claim 1, wherein the amount laquinimod administered is selected from the group consisting of 0.5 mg/day, 1.0 mg/day and 1.5 mg/day.

3. A method of treating a subject afflicted with Huntington's disease comprising administering to the subject an amount of laquinimod so as to thereby treat the subject, wherein the amount laquinimod administered is selected from the group consisting of 0.5 mg/day, 1.0 mg/day and 1.5 mg/day, and wherein laquinimod is preferably laquinimod sodium.

4. The method of any one of claims 1-3, wherein the subject
a) is afflicted with adult onset Huntington's disease;
b) has a Unified Huntington's Disease Rating Scale (UHDRS)−Total Motor Score (TMS) of greater than 5 at baseline;
c) has Unified Huntington's Disease Rating Scale (UHDRS)−Total Functional Capacity (TFC) score of at least 8 at baseline;
d) is ambulatory at baseline;
e) is naïve to a Huntington's disease therapy at baseline; and/or
f) is determined to have ≧36 or 40-49 cytosine-adenosine-guanine (CAG) repeats in the huntingtin gene.

5. The method of claim 4, wherein the subject is naïve to any Huntington's disease therapy or is naïve to laquinimod at baseline.

6. The method of any one of claims 1-5, wherein laquinimod is administered via oral administration.

7. The method of any one of claims 1-6, wherein laquinimod is administered periodically or daily, preferably daily at the same time of the day.

8. The method of claim 7, wherein laquinimod is administered periodically for 12 months or more.

9. The method of any one of claims 1-8, further comprising administration of a second agent for the treatment of Huntington's disease.

10. A package comprising:
a) a pharmaceutical composition comprising one or more unit doses, each such unit dose comprising 0.5-1.5 mg of laquinimod; and
b) instruction for use of the pharmaceutical composition to delay disease progression in a subject afflicted with Huntington's disease.

11. The package of claim 10, wherein the amount of laquinimod in the pharmaceutical composition is selected from the group consisting of 0.5 mg, 1.0 mg and 1.5 mg.

12. A package comprising:
a) a pharmaceutical composition comprising one or more unit doses, each such unit dose comprising 0.5, 1.0 or 1.5 mg of laquinimod; and
b) instruction for use of the pharmaceutical composition to treat a subject afflicted with Huntington's disease.

13. The package of any one of claims 10-12, wherein the package comprises a second pharmaceutical composition comprising an amount of a second agent for the treatment of Huntington's disease.

14. The package of any one of claims 10-13, wherein the pharmaceutical composition is in a solid form, in liquid form, in capsule form or in tablet form.

15. The package of claim 14, wherein the tablet is coated with a coating which inhibits oxygen from contacting the core, preferably the coating comprises a cellulosic polymer, a detackifier, a gloss enhancer, or pigment.

16. The package of any one of claims 10-15, wherein the pharmaceutical composition further comprises mannitol, an alkalinizing agent, an oxidation reducing agent, a lubricant and/or a filler.

17. The package of claim 15, wherein
a) the alkalinizing agent is meglumine;
b) the lubricant is sodium stearyl fumarate or magnesium stearate; and/or
c) the filler is lactose, lactose monohydrate, starch, isomalt, mannitol, sodium starch glycolate, sorbitol, lactose spray dried, lactose anhydrouse, or a combination thereof.

18. The package of any one of claims 10-17, wherein the pharmaceutical composition is stable and free of an alkalinizing agent or an oxidation reducing agent, preferably the pharmaceutical composition is free of an alkalinizing agent and free of an oxidation reducing agent.

19. The package of any one of claims 10-18, further comprising a desiccant, preferably the desiccant is silica gel.

20. The package of any one of claims 10-19, wherein the package is a sealed packaging having a moisture permeability of not more than 15 mg/day per liter, optionally the sealed package comprises an HDPE bottle.

21. The package of claim 20, wherein the sealed package is a) a blister pack in which the maximum moisture permeability is no more than 0.005 mg/day or b) a bottle, preferably closed with a heat induction liner.

22. The package of claims 20 or 21, wherein the sealed package comprises an oxygen absorbing agent, preferably the oxygen absorbing agent is iron.

23. The package of any one of claims 10-22, wherein the pharmaceutical composition is formulated for oral administration and/or formulated for daily administration.

24. The package of any one of claims 10-23, for use in treating or delaying disease progression in a subject afflicted with Huntington's disease.

25. A therapeutic package for dispensing to, or for use in dispensing to, a subject afflicted with Huntington's disease, which comprises:
a) one or more unit doses, each such unit dose comprising 0.5-1.5 mg of laquinimod, and
b) a finished pharmaceutical container therefor, said container containing said unit dose or unit doses, said container further containing or comprising labeling directing the use of said package in delaying disease progression in said subject.

26. The therapeutic package of claim 25, each unit dose comprises an amount of laquinimod selected from the group consisting of 0.5 mg, 1.0 mg and 1.5 mg.

27. A therapeutic package for dispensing to, or for use in dispensing to, a subject afflicted with Huntington's disease, which comprises:
a) one or more unit doses, each such unit dose comprising 0.5 mg, 1.0 mg or 1.5 mg of laquinimod, and
b) a finished pharmaceutical container therefor, said container containing said unit dose or unit doses, said container further containing or comprising labeling directing the use of said package in treating said subject.

28. The therapeutic package of any one of claims 25-27, wherein the package comprises an amount of a second agent for the treatment of Huntington's disease.

29. A pharmaceutical composition comprising one or more unit doses, each such unit dose comprising 0.5-1.5 mg of laquinimod, for use in delaying disease progression in a subject afflicted with Huntington's disease, wherein laquinimod is preferably laquinimod sodium.

30. The pharmaceutical composition of claim 29, comprising an amount of laquinimod selected from the group consisting of 0.5 mg, 1.0 mg and 1.5 mg.

31. A pharmaceutical composition comprising one or more unit doses, each such unit dose comprising 0.5 mg, 1.0 mg and 1.5 mg of laquinimod, for use in treating a subject afflicted with Huntington's disease, wherein laquinimod is preferably laquinimod sodium.

32. The pharmaceutical composition of any one of claims 29-31, comprising an amount of a second agent for the treatment of Huntington's disease.

33. The pharmaceutical composition of any one of claims 29-32, in a solid form, in liquid form, in capsule form or in tablet form.

34. The pharmaceutical composition of claim 33, wherein the tablet is coated with a coating which inhibits oxygen from contacting the core, preferably the coating comprises a cellulosic polymer, a detackifier, a gloss enhancer, or pigment.

35. The pharmaceutical composition of any one of claims 29-34, further comprising mannitol, an alkalinizing agent, an oxidation reducing agent, a lubricant and/or a filler.

36. The pharmaceutical composition of claim 35, wherein
a) the alkalinizing agent is meglumine;
b) the lubricant is sodium stearyl fumarate or magnesium stearate; and/or
c) the filler is lactose, lactose monohydrate, starch, isomalt, mannitol, sodium starch glycolate, sorbitol, lactose spray dried, lactose anhydrouse, or a combination thereof.

37. The pharmaceutical composition of any one of claims 29-36, which is free of an alkalinizing agent or an oxidation reducing agent, preferably the pharmaceutical composition is free of an alkalinizing agent and free of an oxidation reducing agent.

38. The pharmaceutical composition of any one of claims 29-37, formulated for oral administration and/or formulated for daily administration.

39. A package comprising:
a) a pharmaceutical composition of any one of claims 29-38; and
b) instruction for use of the pharmaceutical composition to treat or delay disease progression in a subject afflicted with Huntington's disease.

40. Laquinimod for the manufacture of a medicament for use in delaying disease progression in a subject afflicted Huntington's disease, wherein the medicament comprises one or more unit doses, each such unit dose comprising 0.5-1.5 mg of laquinimod.

41. Laquinimod for the manufacture of a medicament for use in treating a subject afflicted Huntington's disease, wherein the medicament comprises one or more unit doses, each such unit dose comprising 0.5, 1.0 or 1.5 mg of laquinimod.
